(19) United States
(12) Patent Application Publication
(10) Pub. No.: US 2009/0292161 A1
(43) Pub. Date: Nov. 26, 2009

(54) MULTI-MODE HEARING PROSTHESIS

(75) Inventor: John L. Parker, Roseville (AU)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove (AU)

(21) Appl. No.: 12/353,957

(22) Filed: Jan. 14, 2009

Related U.S. Application Data
(60) Provisional application No. 61/041,185, filed on Mar. 31, 2008.

(51) Int. Cl. A61F 11/04; H04R 25/00

(52) U.S. Cl. 600/25; 381/326

(57) ABSTRACT

A multi-mode hearing prosthesis for enhancing the hearing of a recipient, comprising: a sound input element configured to receive a sound signal component; a frequency spectral analysis module configured to analyze the sound signal component and to categorize the component into at least a high- or lower-frequency component; a bone conduction processor configured to generate bone conduction stimulation signals from at least one of said high- and lower-frequency component for bone conduction stimulation of the recipient's skull; and a second stimulation processor configured to generate auditory stimulation signals from at least one of said high- and lower-frequency components for stimulating the recipient.

### CROSS-REFERENCE TO RELATED APPLICATIONS

The present application claims the benefit of U.S. Provisional Patent Application 61/041,185; filed Mar. 31, 2008, which is hereby incorporated by reference herein.

### FIELD OF THE INVENTION

The present invention relates generally to a hearing prosthesis, and more particularly, to a multi-mode hearing prosthesis.

### RELATED ART

Hearing loss, which may be due to many different causes, is generally of two types, conductive or sensorineural. In many people who are profoundly deaf, the reason for their deafness is sensorineural hearing loss. This type of hearing loss is due to absence, destruction, or damage to the hair cells that transduce acoustic signals into nerve impulses in the cochlea. Various hearing prostheses have been developed to provide individuals who suffer from sensorineural hearing loss with the ability to perceive sound. One type of hearing prosthesis, commonly referred to as a cochlear implant, electrically stimulates the auditory nerve via an electrode array implanted in the cochlea to induce a hearing percept in the prosthesis recipient.

Conductive hearing loss occurs when the normal mechanical pathways which conduct sound to hair cells in the cochlea are impeded. This problem may arise, for example, from damage to the ossicular chain. Individuals who suffer from conductive hearing loss frequently still have some form of residual hearing because the hair cells in the cochlea are often undamaged. For this reason, individuals who suffer from conductive hearing loss are typically not candidates for a cochlear implant, because insertion of the electrode array into a cochlea results in the severe damage or destruction of the most of the hair cells within the cochlea.

The type of hearing prosthesis commonly suggested to individuals suffering from conductive hearing loss is the acoustic hearing aid. Hearing aids receive ambient sound via the outer ear, amplify the sound, and direct the amplified sound into the ear canal. The amplified sound reaches the cochlea and causes motion of the cochlea fluid (perilymph), thereby stimulating the hair cells in the cochlea.

Hearing loss may not be complete in all sufferers, and also may not be entirely sensorineural or conductive. For example, as people age they frequently experience progressive sensorineural hearing loss. Usually this loss is more prevalent and more severe at higher frequencies. Thus, it is estimated that a large segment of the hearing-impaired population exhibits sensorineural hearing loss relative to high frequency sounds, but maintains the ability to transduce middle-to-lower frequency sounds through functioning hair cells. The usual method to restore this high frequency hearing loss is by using a hearing aid that increases the amplitude of the acoustic energy applied to the tympani membrane.

Unfortunately, hearing aids are not always ideal for all individuals who may have some residual hearing. For example, some individuals are prone to chronic inflammation or infection of the ear canal and cannot wear hearing aids. Other individuals have malformed or absent outer ear and/or ear canals as a result of a birth defect, or as a result of common medical conditions such as Treacher Collins syndrome or Microtia. Furthermore, the dramatic increase in acoustic amplitudes that are sometimes necessary in order for the sufferer to hear the higher frequencies can further degrade residual hearing, resulting in a further decrease in the ability to hear the higher frequencies.

Similarly, hearing prostheses such as cochlear implants alone may also not be ideal for all individuals who may have residual hearing since the permanent implantation of an electrode array into the components of the ear which still provide residual hearing may permanently damage the organs in that area such that no residual hearing will be left in those areas immediately following implantation or some time after.

Furthermore, traditional hearing aids and other hearing prostheses use a single sound input component which allows the device to detect and deliver sound from only a single sound source. However, for people having normal hearing, they are often able to detect and comprehend, at least to some degree, multiple conversations or sound sources under certain circumstances.

### SUMMARY

According to one aspect of the present invention, there is provided a multi-mode hearing prosthesis for enhancing the hearing of a recipient, comprising: a sound input element configured to receive a sound signal component; a frequency spectral analysis module configured to analyze the sound signal component and to categorize the component into at least a high- or lower-frequency component; a bone conduction processor configured to generate bone conduction stimulation signals from at least one of said high- and lower-frequency component for bone conduction stimulation of the recipient's skull; and a second stimulation processor configured to generate auditory stimulation signals from at least one of said high- and lower-frequency components for stimulating the recipient.

According to another aspect of the present invention, there is provided a multi-mode hearing prosthesis for enhancing the hearing of a recipient, comprising: a first sound input element configured to receive a high-frequency sound signal component; a second sound input element configured to receive a lower-frequency sound signal component; a bone conduction processor, configured to process said high-frequency sound signal component from said first input element and further configured to generate bone conduction stimulation to stimulate the recipient via bone conduction stimulation; and a second stimulation processor configured to process said lower-frequency sound signal component from said second input element and further configured to generate stimulation signals to stimulate the recipient via a second stimulation mode, wherein each of said first and second stimulation processors are configured to process said first and second signal components simultaneously.

According to a further aspect of the present invention, there is provided a method for rehabilitating the hearing of a recipient with a multi-mode hearing prosthesis having two or more stimulation modules, comprising: receiving an electrical signal representative of an acoustic sound signal; analyzing said sound signal to generate at least a high-frequency component and a lower-frequency component from said acoustic sound signal; delivering said high-frequency component via bone conduction to the recipient's skull bone; and deliver said lower-frequency component via acoustic stimulation to the recipient's hearing organ.

According to yet another aspect of the present invention, there is provided a multi-mode hearing prosthesis for enhancing the hearing of a recipient having two or more stimulation modules, comprising: means for receiving an electrical signal representative of an acoustic sound signal; means for analyzing said sound signal to generate at least a high-frequency component and a lower-frequency component from said acoustic sound signal; means for delivering said high-frequency component via bone conduction to the recipient's skull bone; and means for deliver said lower-frequency component via acoustic stimulation to the recipient's hearing organ.

According to a still further aspect of the present invention, there is provided a method of stimulating a recipient with a multi-mode hearing prosthesis, comprising: receiving a high-frequency sound signal component at a first sound input element; receiving a lower-frequency sound signal component at a second sound input element; processing said high-frequency sound signal component with a bone conduction processor configured to generate and deliver bone conduction stimulation; and processing said lower-frequency sound signal component with a second stimulation processor configured to generate and deliver acoustic stimulation via a second stimulation mode, wherein said bone conduction processor and second stimulation processor operate substantially concurrently.

### BRIEF DESCRIPTION OF THE DRAWINGS

Illustrative embodiments of the present invention are described herein with reference to the accompanying drawings, in which:

FIG. 1 is a perspective view of a multi-mode hearing prosthesis provided to a recipient according to one embodiment of the present invention;

FIG. 2A is a high-level functional block diagram of a multi-mode hearing prosthesis according to one embodiment of the present invention, such as the prosthesis of FIG. 1;

FIG. 2B is a detailed functional block diagram of the multi-mode hearing prosthesis illustrated in FIG. 2A;

FIG. 3 is an exploded view of a multi-mode hearing prosthesis according to one embodiment of the present invention;

FIG. 4 is a high-level flowchart illustrating the processing of an input sound into high and low frequency components in a multi-mode hearing prosthesis according to one embodiment of the present invention;

FIG. 5 is a detailed flowchart illustrating the processing in the multi-mode hearing prosthesis illustrated in FIG. 4; and

FIG. 6 is a high-level functional block diagram of a multi-mode hearing prosthesis according to another embodiment of the present invention.

### DETAILED DESCRIPTION

Embodiments of the present invention are generally directed to a multi-mode hearing prosthesis for analyzing a received acoustic sound signal and separating the sound signal into its frequency components such as, for example, high-frequency and low-frequency components. The signal components are provided to various stimulation modules which further processes the received component and transmits them to the recipient. According to one embodiment of the present invention, high-frequency components are provided to a bone conduction stimulation module which converts the received sound signal component into a mechanical force to be delivered via a recipient's skull to the recipient's hearing organs. The multi-mode hearing prosthesis includes a sound input component, such as microphone, to receive the acoustic sound signal, a spectral analysis module configured to analyze and separate the received sound signal into high-frequency and low-frequency components, and two or more stimulation modules such as a bone conduction module and an acoustic stimulation module. The bone conduction module comprises a bone-conduction processor configured to generate an electrical signal representing the received high-frequency signal component, a transducer to convert the signal component into a mechanical force for delivery to the recipient's skull, and one or more anchors implanted into the recipient's skull bone to transmit the mechanical force received from the transducer to the recipient's skull.

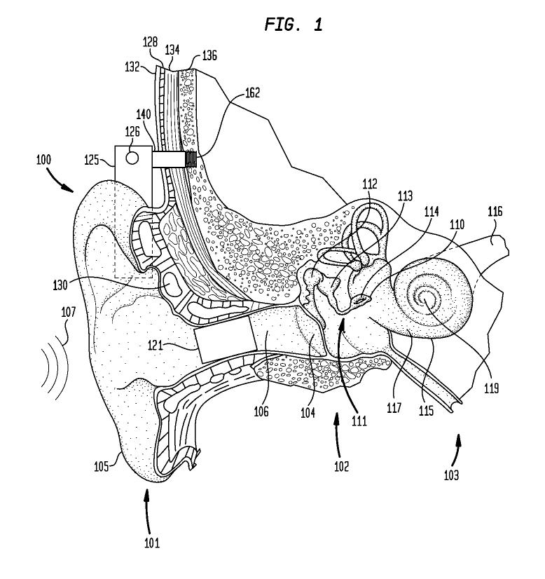

FIG. 1 is a perspective view of an embodiment of a multi-mode hearing prosthesis 100 implementing a vibrational and acoustic stimulation modes according to one embodiment of the present invention. In a fully functional human hearing anatomy, outer ear 101 comprises an auricle 105 and an ear canal 106. A sound wave or acoustic pressure 107 is collected by auricle 105 and channeled into and through ear canal 106. Disposed across the distal end of ear canal 106 is a tympanic membrane 104 which vibrates in response to acoustic wave 107. This vibration is coupled to oval window or fenestra ovalis 110 through three bones of middle ear 102, collectively referred to as the ossicles 111 and comprising the malleus 112, the incus 113 and the stapes 114. Middle ear 102 serves to filter and amplify acoustic wave 107, causing oval window 110 to articulate, or vibrate. Such vibration sets up waves of fluid motion within the perilymph of cochlea 115 of inner ear 103. Such fluid motion, in turn, activates the hair cells (not shown) that line the inside of cochlea 115. Activation of the hair cells causes appropriate nerve impulses to be transferred through the spiral ganglion cells and auditory nerve 116 to the brain (not shown), where they are perceived as sound.

Within cochlea 115, maximum excitation of the hair cells occurs along the basilar membrane (not shown), which is a membrane that separates certain internal canals running along the substantial length of cochlea 115. The position of the excitation along this basilar membrane determines the perception of pitch and loudness according to the place theory. Due to this anatomical arrangement, cochlea 115 has characteristically been referred to as being “tonotopically mapped.” That is, regions of cochlea 115 toward basal region 117 are responsive to high frequency signals, while regions of cochlea 115 toward the apical end 119 are responsive to low frequency signals. These tonotopical properties of cochlea 115 are exploited in a cochlear implant by delivering stimulation signals within a predetermined frequency range to a region of the cochlea that is most sensitive to that frequency range.

The different stimulation modes are each implemented in a respective series of components collectively referred to herein as a stimulation modules. For example, the multi-mode hearing prosthesis 100 provides vibrational stimulation generated by a bone conduction hearing module housed at least partially within housing 125 via implanted anchor 162 and the acoustic stimulation is generated by an acoustic hearing module 121. stimulation and acoustic stimulation. The vibrational stimulation FIG. 1 illustrates an example of the positioning of multi-mode hearing prosthesis 100 relative to outer ear 101, middle ear 102 and inner ear 103 of a recipient of hearing prosthesis 100. Because they may share components, the stimulation module may not have the same components as the hearing prostheses conventionally provided the same type of stimulation.

In the embodiments illustrated in FIG. 1, multi-mode hearing prosthesis 100 comprises a housing 125 with a microphone (not shown) positioned therein or thereon. Housing 125 is coupled to the body of the recipient via an anchoring system comprising coupling 140 and implanted anchor 162. As described below, multi-mode hearing prosthesis 100 may comprise a spectral analysis module and two or more stimulation modules, for example, a bone conduction stimulation module. The bone conduction stimulation module may comprise a bone-conduction processor, a transducer, transducer drive components, an anchoring system, and/or various other circuits/components. The anchor system may be fixed to bone 136. In various embodiments, the anchor system may be surgically placed through skin 132, muscle 134 and/or fat 128. In certain embodiments, the anchor system may comprise a coupling 140 and one or more anchoring elements 162. Also, in one embodiment of the present invention, an acoustic amplification module may comprise an acoustic amplification module and a speaker 121 for outputting an amplified acoustic sound.

The spiral ganglion cells that are responsible for the perception of high frequency sounds are generally located at the basal end of the cochlea 115, i.e., that end of the cochlea closest to the oval window 110. For those individuals who suffer from high frequency hearing loss, the hair cells in the basal region of the cochlea are ineffective or otherwise damaged to the point where it is not possible to activate them. Hence, in accordance with one embodiment of the present invention, a multi-mode hearing prosthesis 100 is positioned proximate to and retained by outer ear 101. Anchor 162 for the bone conduction module (not shown) is coupled to multi-mode hearing prosthesis 100 and implanted in bone 136. The microphone signals are amplified and processed by an amplification module (not shown) in multi-mode hearing prosthesis 100.

In certain embodiments of the present invention, the transducer may comprise a piezoelectric element. The piezoelectric element converts an electrical signal applied thereto into a mechanical deformation (i.e. expansion or contraction) of the element. The amount of deformation of a piezoelectric element in response to an applied electrical signal depends on material properties of the element, orientation of the electric field with respect to the polarization direction of the element, geometry of the element, etc.

The deformation of the piezoelectric element may also be characterized by the free stroke and blocked force of the element. The free stroke of a piezoelectric element refers to the magnitude of deformation induced in the element when a given voltage is applied thereto. Blocked force refers to the force that must be applied to the piezoelectric element to stop all deformation at the given voltage. Generally speaking, piezoelectric elements have a high blocked force, but a low free stroke. In other words, when a voltage is applied to the element, the element will can output a high force, but will only produce a small stroke.

In some piezoelectric transducers, the maximum available transducer stroke is equivalent to the free stroke of the piezoelectric element. As such, some multi-mode hearing prostheses utilizing these types of piezoelectric transducer have a limited transducer stroke and corresponding limits on the magnitude of the mechanical force that may be provided to the skull.

The acoustic stimulation module comprises an acoustic amplification processor, which is configured to amplify (positively or negatively) the received low-frequency component, and a speaker positioned sufficiently proximate to the recipient's hearing organs such that the amplified low-frequency component can be perceived by the recipient's residual hearing.

The in-the-canal hearing aid and its speaker or other output module 121 may be of conventional design and may be configured to receive and amplify the lower frequency components received, thereby presenting amplified acoustic waves (not shown) to tympanic membrane 104. Other designs for output module 121 may also be used in other embodiments of the present invention.

For example, embodiments of the present system may be beneficially comprise an acoustic output module in which speaker component 121 does not occlude ear canal 106. Unlike traditional hearing aids or other systems using an acoustic output component, stimulation for high frequency sound components are generated and delivered by a non-acoustic amplification module, thus avoiding acoustic feedback which have been problematic with certain past systems. Instead, under embodiments of the present invention, high-frequency sound components can be directed to the basilar region of cochlea 115, while only low-frequency sound components will be emitted by acoustic amplification output module 121, thus avoiding feedback of high-frequency sound components. It is to be understood that output module 121 may be formed and configured to occlude ear canal 106, or may be made (for example, as a cylinder having a hollow center) so as to allow the passing of air, sound, moisture, etc. through acoustic output module 121.

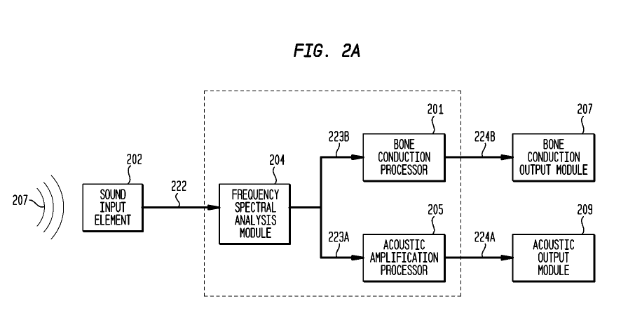

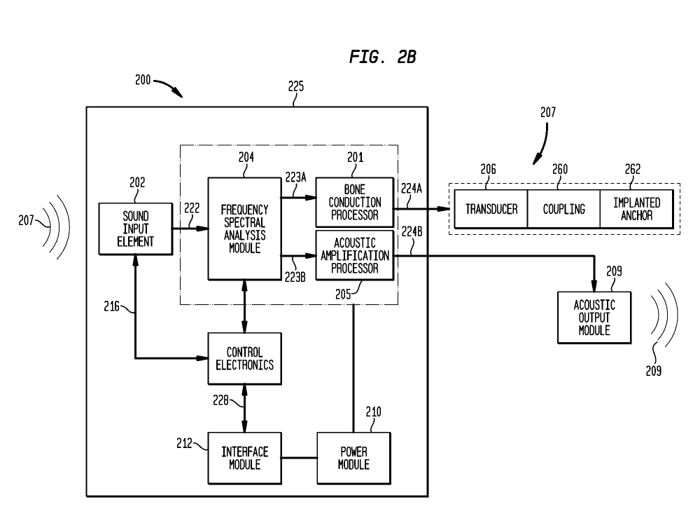

FIG. 2A is a high-level functional block diagram of a multi-mode hearing prosthesis according to one embodiment of the present invention as illustrated in FIG. 2A. In the illustrated embodiment, a sound 207 is received by a sound input element 202. In certain embodiments of the present invention, sound input element 202 is a microphone configured to receive sound 207, and to convert sound 207 into an electrical signal 222. As described below, in other embodiments sound 207 may be received by sound input element 202 already in the form of an electrical signal.

As shown in FIG. 2A, according to one embodiment of the present invention, sound input element 202 receives sound 207 and outputs electrical signal 222, which comprises a series of sound components (also referred to as sound components 222), to a frequency spectral analysis module 204. Frequency spectral analysis module 204 is configured to analyze components 222 of the electrical signal representing components of sound 207, and to categorize signal components 222 into high-frequency components 223B and lower-frequency components 223A. Those categorized components 223A, 223B are then sent to other circuits for further processing. For example, in one embodiment of the present invention, bone conduction processor 201 receives higher-frequency components 223B for further processing and conversion into bone conduction control signals 224B to be output by bone conduction output module 207. Acoustic amplification processor 205 receives lower-frequency components 223A for further processing and conversion into an amplified acoustic signal 224A to be output by acoustic output module 209. It is to be understood that although two components (high and lower) are discussed above, the present invention may separate signal 222 into other components, for example mid-frequency components, in addition to high- and low-frequency components, for processing and ultimately transmitting by other or the same modules discussed herein.

In the embodiment of the present invention described immediately above, frequency spectral analysis module 204 and processors 201 and 205 are housed in a common housing. However, in other embodiments of the present invention, each of the processors 201, 205 may each be housed with their respective output modules 207, 209. For example, in one embodiment of the present invention, bone conduction processor 201 may be housed separate from frequency spectral analysis module 204, and housed instead with bone conduction output module 207. Furthermore, it is to be understood that in other embodiments of the present invention, processors 201, 205 (and any other stimulation-specific processors) may be housed in a single housing, apart from the housing containing frequency spectral analysis module 204.

Stimulation-specific processors (e.g., bone conduction processor 201, acoustic amplification processor 205) are configured to provide additional processing on the received signals 223A, 223B. Such further processing may include, but is not limited to, applying one or more stimulation strategies, additional amplification, optimization, smoothing, and filtering. It is to be understood that such further processing may also be performed before frequency spectral analysis 204, or before the signals 223A, 223B are sent to the selected stimulation-specific processors 201, 205. In one embodiment of the present invention, a separate smoothing circuit (not shown) may be provided to allow a smooth, seamless transition from the acoustic enhancement provided for low-to-middle frequencies and the bone conduction stimulation provided for the high frequency components of sound 207.

In addition to the components described above with reference to FIG. 2A, FIG. 2B illustrates other components present in other embodiments of the present invention. As shown, FIG. 2B also illustrates a power module 210. Power module 210 provides electrical power to one or more components of multi-mode hearing prosthesis 200. For ease of illustration, power module 210 has been shown connected only to interface module 212 and frequency spectral analysis module 204. However, it should be appreciated that power module 210 may be used to supply power to any electrically powered circuits/components of multi-mode hearing prosthesis 200.

In the embodiment illustrated in FIG. 2B, sound pickup device 202, frequency spectral analysis module 204, power module 210, interface module 212, and the control electronics have all been shown as integrated in a single housing, referred to as housing 225. However, it should be appreciated that in certain embodiments of the present invention, one or more of the illustrated components may be housed in separate or different housings. Similarly, it should also be appreciated that in such embodiments, direct connections between the various modules and devices are not necessary and that the components may communicate, for example, via wireless connections.

Bone conduction output module 207, according to one embodiment of the present invention, comprises coupling 260, transducer 206 and implanted anchor 262. Coupling 260 is configured to provide a mechanical connection between transducer 206 and implanted anchor 262. Transducer 206 generates an output force for transmission to the skull of the recipient. This force is communicated to the recipient's skull via anchor 262. As shown in FIG. 2B, coupling 260 is attached to transducer 206 and vibration is received directly therefrom. In other embodiments, coupling 260 is attached to housing 225, where transducer 206 is physically connected to housing 225 but not directly connected to coupling 260, and vibration generated by the remotely-located transducer 206 is applied through housing 225 to coupling 260. According to one embodiment of the present invention, the vibration received by coupling 260 from transducer 206 causes coupling 260 to vibrate. Since, according to this embodiment of the present invention, coupling 260 is mechanically coupled to bone anchor 262, bone anchor 262 also vibrates. The vibration, communicated from coupling 260 to bone anchor 262 mechanically is then transferred from bone anchor 262 to the recipient's bone 136.

In addition to mechanical couplings between bone anchor 262 described above, certain embodiments of the present invention may also utilize other types of couplings between bone anchor 262 and transducer 206 or housing 225. For example, bone anchor 262 may be magnetically coupled to housing 225 or to transducer 206 such that the mechanical forces generated by transducer 206 are transmitted magnetically to bone anchor 262. Furthermore, although transducer 206 and bone anchor 262 have been presently described as two separate components, it is to be understood that transducer 206 and bone anchor 262 as described herein may be manufactured as a single or unitary component or manufactured separately and permanently joined together.

Also in the embodiment shown in FIG. 2B, acoustic amplification processor 205 is configured to receive lower-frequency components 223B and to deliver amplified, either positively or negatively, acoustic signal 224B to acoustic output module 209. In one embodiment of the present invention, acoustic output module 209 may comprise a speaker or other acoustic output element capable to providing acoustic stimulation to the recipient as in a traditional hearing aid system. In other embodiments of the present invention, acoustic stimulation amplification processor 205 may be configured to amplify (both to enlarge or to reduce or muffle) the signal component appropriately, based on the residual hearing capabilities of the recipient. In one embodiment of the present invention, multi-mode hearing prosthesis 100 may be fitted for the recipient such that the lower-frequency sensitivity of the recipient can be determined or factored into the fitting program provided to the multi-mode hearing prosthesis 100. Acoustic amplification processor 205 may comprise other components configured to provide additional benefits for the recipient. For example, in one embodiment of the present invention, acoustic amplification processor 205 may be configured with a smoothing circuit configured to provide a smoothed acoustic stimulation that is free of sudden spikes or valleys in acoustic stimulation which can be uncomfortable at least, or even harmful to the hearing organs of the recipient.

Multi-mode hearing prosthesis 200 may further comprise an interface module 212. Interface module 212 includes one or more components that allow the recipient to provide inputs to, or receive information from, elements of multi-mode hearing prosthesis 200.

As shown, control electronics 246 may be connected to one or more of interface module 212, sound pickup device 202, frequency spectral analysis module 204, and one or both processors 201, 205. In embodiments of the present invention, based on inputs received at interface module 212, control electronics 246 may provide instructions to, or request information from, other components of multi-mode hearing prosthesis 200. In certain embodiments, in the absence of user inputs, control electronics 246 control the operation of multi-mode hearing prosthesis 200.

Although embodiments of the present invention have been described above as using stimulation modules including a bone conduction processor 201 or output module 207, or an acoustic processor 205 and output module 209, it is to be understood that in other embodiments of the present invention, other stimulation processors and/or output modules may be used instead of, or in addition to, the stimulation modules already described in further embodiments of the present invention. For example, in one embodiment of the present invention, a direct acoustic stimulator module may be incorporated into the multi-mode hearing prosthesis of the present invention. With such a hearing prosthesis, the direct acoustical stimulator (not shown) has a proximal end connected to a stimulator unit via electrical wiring, and a stimulation rod at a distal end implanted in the recipient's cochlea. The stimulation rod may be configured to be inserted into the round window and is configured to directly interact with cochlear fluids. In such an embodiment, the direct stimulation rod is mechanically coupled to a vibrator or transducer which generates and communicates mechanical vibration to the stimulation rod, which in turn interacts with and causes the cochlear fluids to be physically moved. This physical movement of cochlear fluids in turn causes the fine hair in the cochlea to move, thus providing some or full hearing sensation to the recipient. As described above with respect to bone conduction stimulation and acoustic stimulation, this direct acoustic stimulation method may be used in conjunction with other stimulation methods in order to supplement or better customize the prosthetic stimulation provided to the recipient. In one embodiment of the present invention, direct acoustic stimulation may be used to provide stimulation for high-frequency sound component 223A, while acoustic amplification methods can be used for lower-frequency sound component 223B. In yet further embodiments of the present invention, direct acoustic stimulation can be used for lower-frequency signal component 223B while high-frequency stimulation can be processed by bone conduction processor 201. Further details of a direct acoustical stimulation device may be found in commonly-owned and co-pending application, entitled “Device for Direct Acoustical Stimulation of the Inner Ear”, filed concurrently herewith and incorporated by reference.

Other stimulation methods and associated processors and output modules may also be used in further embodiments of the present invention. In one such embodiment of the present invention, the recipient's middle-ear, specifically the mastoid bone, is mechanically coupled to a middle-ear stimulation output module (not shown) which generates vibration or other mechanical forces and communicates that vibration or forces to the mastoid bone, which in turn communicates the vibration or forces to the cochlea, including the cochlear fluids and fine hair cells therein, in order to generate auditory sensation for the recipient. In other embodiments of the present invention in which the middle-ear or its mastoid bone is thus vibrated, an extension or arm may be coupled to a transducer or other vibration producing element in order to communicate vibration or forces generated at a location remote from the mastoid bone to the mastoid bone, thus allowing for an alternative or optimal location for the vibration element while also optimizing or effectively communicating the vibratory forces to the middle-ear and the mastoid bone found therein. Further details of a middle-ear stimulation device may be found in commonly-owned and co-pending application, entitled “An Implantable Cochlear Access Device”, filed concurrently herewith and incorporated by reference.

In embodiments of the present invention, transducer 206 may be one of many types and configurations of transducers, now known or later developed. In one embodiment of the present invention, transducer 206 may comprise a piezoelectric element which is configured to deform in response to the application of electrical signal 224. Piezoelectric elements that may be used in embodiments of the present invention may comprise, for example, piezoelectric crystals, piezoelectric ceramics, or some other material exhibiting a deformation in response to an applied electrical signal. Exemplary piezoelectric crystals include quartz (SiO2), Berlinite (AlPO4), Gallium orthophosphate (GaPO4) and Tourmaline. Exemplary piezoelectric ceramics include barium titanate (BaTiO30), lead zirconate titanate (PZT), or zirconium (Zr).

Some piezoelectric materials, such as barium titanate and PZT, are polarized materials. When an electric field is applied across these materials, the polarized molecules align themselves with the electric field, resulting in induced dipoles within the molecular or crystal structure of the material. This alignment of molecules causes the deformation of the material.

In other embodiments of the present invention, other types of transducers may be used. For example, various motors configured to operate in response to electrical signal 224 may be used.

Transducer 206 is configured to generate substantially lateral mechanical forces that are parallel to the surface of recipient's skull 136. Transducer 206 is coupled to one or more implanted anchors (not shown), also referred to as “bone anchors”, mechanically coupled to transducer 206, and as a result the bone anchor receive forces exerted by transducer 206 in opposite directions. Delivery of this output force causes one or more of motion or vibration of the recipient's skull, thereby activating the hair cells in the cochlea via cochlea fluid motion. While the recipient's skull, particularly in the components thereof in the area where the bone anchor are implanted, are caused to bend, flex, move, vibrate, or otherwise change its position because of the forces transferred via the bone anchor moving in opposite directions from one other, the multi-mode hearing prosthesis 200 produces no net rotation or translation force on the recipient's head.

In certain embodiments of the present invention, frequency spectral analysis module 204 includes a printed circuit board (PCB) to electrically connect and mechanically support the components of frequency spectral analysis module 204. Sound input element 202 may comprise one or more microphones (not shown) and is attached to the PCB.

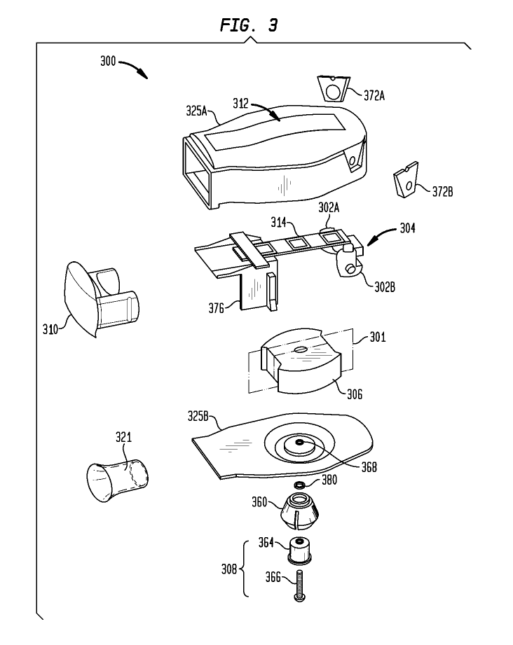

FIG. 3 illustrates an exploded view of one embodiment of multi-mode hearing prosthesis 200 of FIGS. 2A and 2B, referred to herein as multi-mode hearing prosthesis 300. As shown, multi-mode hearing prosthesis 300 comprises an embodiment of electronics module 204, referred to as electronics module 304. As explained above, included within electronics module 304 are a signal processor, an output module, and control electronics. For ease of illustration, these components have not been illustrated in FIG. 3.

In the illustrated embodiment, electronics module 304 includes a printed circuit board 314 (PCB) to electrically connect and mechanically support the components of electronics module 304. A plurality of sound input elements are attached to PCB 314, shown as microphones 302A and 302B to receive a sound.

In one embodiment two microphones 302A and 302B are provided. Preferably, each microphone is positioned equidistant or substantially equidistant from the longitudinal axis of the hearing prosthesis; however, microphones 302A and 302B may be positioned in any suitable position. By being positioned equidistant or substantially equidistant from the longitudinal axis, multi-mode hearing prosthesis 300 can be used on either side of a patient's head. The microphone facing the front of the recipient is generally chosen using the selection circuit as the operating microphone, so that sounds in front of the recipient can be heard; however, the microphone facing the rear of the recipient can be chosen, if desired. It is noted that it is not necessary to use two or a plurality of microphones and only one microphone may be used in any of the embodiments described herein.

In the embodiment illustrated in FIG. 3, multi-mode hearing prosthesis 300 further comprises a battery shoe 310 for supplying power to components of hearing prosthesis 300. Battery shoe 310 may include one or more batteries. In certain embodiments, PCB 314 is attached to a connector 376. Connector 376 is configured to mate with battery shoe 310. In certain embodiments, connector 376 and battery shoe 310 may be releasably snap-locked to one another. Furthermore, in such embodiments, one or more battery connects (not shown) are disposed in connector 376 to electrically connect battery shoe 310 with electronics module 304.

In the embodiment illustrated in FIG. 3, multi-mode hearing prosthesis 300 further includes a two-part housing 325, comprising first housing component 325A and second housing component 325B. Housing components 325 are configured to mate with one another to substantially seal multi-mode hearing prosthesis 300.

In the embodiment of FIG. 3, first housing component 325A has an opening therein for receiving battery shoe 310. In such embodiments, battery shoe protrudes through first housing component 325A and may be removed or inserted by the recipient. Also in the illustrated embodiment, microphone covers 372 can be releasably attached to first housing component 325A. Microphone covers 372 can provide a barrier over microphones 302 to protect microphones 302 from dust, dirt or other debris.

Multi-mode hearing prosthesis 300 further can include an embodiment of interface module 212, referred to herein as interface module 312. Interface module 312 is configured to provide or receive recipient inputs from the recipient.

Also as shown in FIG. 3, in the embodiment shown, multi-mode hearing prosthesis 300 comprises bone conduction output module 207, referred to as bone conduction module 306. Bone conduction module 306 comprises a transducer (not shown) that generates an output force that causes movement of the cochlea fluid so that a sound may be perceived by the recipient. The output force may result in mechanical vibration of the recipient's skull, or in physical movement of the skull about the neck of the recipient. As noted above, in certain embodiments, multi-mode hearing prosthesis 300 delivers the output force to the skull of the recipient via an implanted anchor 362. Anchor 362 is mechanically coupled to coupling 260, illustrated in FIG. 3 as coupling 360. In the embodiment illustrated in FIG. 3, coupling 360 is configured to be attached to second housing component 325B. As such, in this embodiment, vibration from transducer 306 is provided to coupling 360 through housing 325B. In the embodiment shown in FIG. 3, an opening 368 is provided in second housing component 325B. A screw (not shown) may be inserted through opening 368 to attach transducer 306 to coupling 360. In such embodiments, an O-ring 380 may be provided to seal opening 368 around the screw.

As noted above, anchor 362 comprises a bone screw 366 implanted in the skull of the recipient and an abutment 364. In an implanted configuration, screw 366 protrudes from the recipient's skull through the skin. Abutment 364 is attached to screw 366 above the recipient's skin. In other embodiments, abutment 364 and screw 366 may be integrated into a single implantable component. Coupling 360 is configured to be releasably attached to abutment 364 to create a vibratory pathway between transducer 306 and the skull of the recipient.

FIG. 3 also illustrates a speaker 306 which, though not shown as being coupled, is communicably coupled to PCB 314 and configured to output an amplified sound signal generated by acoustic output module 209 of dual-mode hearing prosthesis 300 to the recipient, in a manner similar to that of traditional hearing aids. As noted elsewhere in this disclosure, embodiments of the present invention are configured such that lower frequency component 223B of the received input sound 207 are amplified and directed ultimately to speaker 306 while the subset representing the high-frequency component 223A of the received input sound are processed and directed ultimately to transducer 306 for communicating the higher frequency component by means of bone conduction to the recipient.

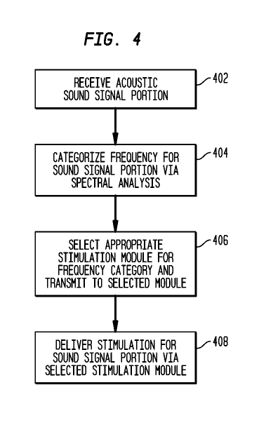

FIG. 4 illustrates a high level block diagram, illustrating the selection and stimulation using one of multiple stimulation modes available according to the one embodiment of a multi-mode hearing prosthesis of the present invention. At block 402, multi-mode hearing prosthesis 200 receives a component 222 of an acoustic sound signal. In certain embodiments, the acoustic sound signal is received via a single microphone. In other embodiments, the acoustic sound signal is received via multiple microphones. In yet further embodiments of the present invention, the input sound is received via an electrical input. In still other embodiments, a telecoil integrated in, or connected to, multi-mode hearing prosthesis 200 may be used to receive the acoustic sound signal.

At block 404, the acoustic sound signal component received by multi-mode hearing prosthesis 200 is categorized by frequency spectral analysis module 204 as being a high or low frequency signal component 223A, 223B. Frequency spectral analysis module 204 may also provide additional processing of the received sound signal component, for example eliminating background or other unwanted noise signals received by multi-mode hearing prosthesis 200.

At block 406, the appropriate stimulation module for the categorized frequency components 223A, 223B is selected, and the categorized frequency component 223A or 233B is transmitted or otherwise provided to their respective stimulation processors, such as for example bone conduction processor 201 or acoustic amplification processor 205. At block 408, the selected stimulation output module delivers the processed signal 224A or 224B to the recipient. It is to be understood that signal components as used herein refers to the particular segment or part of the sound signal being considered for processing and ultimately delivery to the recipient. As one of skill in the art would recognize, a perceived sound, especially in the digital processing context, can be seen as a stream of separate sound or signal segments or components. As the multi-mode hearing prosthesis of the present invention is intended to receive, process and provide signals representing the received sound signal in a live or substantially real-time manner, the received sound signal may be viewed as comprising many different segments or components. The components may consist of the sound perceived or detected during fixed-period windows of time set to the average frequency detected during each fixed-period window of time. Alternatively, the components 222 may represent a received signal that is substantially the same frequency for a determined length of time, the duration and frequency information being captured and transmitted downstream in the system, and used to ultimately process and provided stimulation to the recipient as described herein.

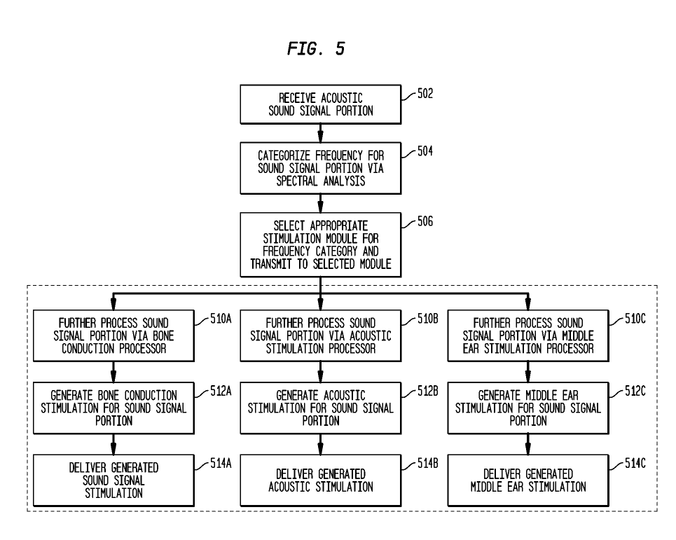

FIG. 5 illustrates a detailed block diagram of one embodiment of the present invention as illustrated and described with respect to FIG. 4. In the particular embodiment illustrated, sound signal components 222 are received 502 and categorized 504 into high-, mid- and low-frequency components 223. The stimulation module appropriate for each of the high-, mid- and low-frequency components is selected 506, and the signal component is transmitted 506 or otherwise provided to the selected stimulation module. In the embodiment of the present invention illustrated, high-frequency components are provided 510A to the bone conduction process (not shown) for further processing and to generate 512A the bone conduction stimulation. The bone conduction stimulation is then delivered 514A to the recipient, who perceives the high-frequency sound signal component. In this embodiment, mid-frequency signal components are provided 510B to the acoustic stimulation processor (not shown), which generates 512B the acoustic stimulation and then delivers 514B the generated acoustic stimulation to the recipient. Furthermore, low-frequency signal components are provided 510C to the middle ear stimulation processor (not shown), as described above, which generates 512C the middle ear stimulation and delivers 514C the generated middle ear stimulation to the recipient. Thus, multiple stimulation modules are used to provide different frequency category sound signal components via different stimulation methods and modules, according to one embodiment of the present invention.

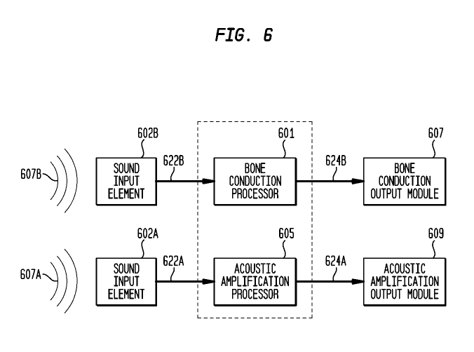

In a further embodiment of the present invention, two or more sound input elements 620A, 620B, such as microphones, are used. In a particular embodiment, each of microphones 620A, 620B are configured to output signals within a particular frequency range. For example, in the embodiment illustrated in the function block diagram depicted in FIG. 6, sound signal component 622A output from microphone 620A is a lower-frequency signal component, and sound signal component 622B output from microphone 620B is a high-frequency signal component. As described above with respect to other embodiments of the present invention, lower-frequency component 622A is processed by acoustic amplification processor 605, and the amplified acoustic signal 624A is provided to acoustic amplification output module 609 for acoustic stimulation of the recipient's hearing organs. Furthermore, high-frequency component 622B is processed by bone conduction processor 601, and the bone conduction stimulation signal 624A is provided to bone conduction output module 607 for bone conduction stimulation of the recipient's hearing organs.

In other embodiments of the present invention incorporating two sound input elements 602A, 602B, multiple sound sources, occurring at substantially different frequency rates, for example low-frequency speech and high-frequency music at a symphony or concert, may both be detected by the sound input elements 602A and 602B, and both be processed and ultimately provided to the recipient. This provision of sounds from multiple sound sources is already possible in individuals having normal hearing where the sound sources operate at sufficiently different frequency ranges. Where the frequency ranges are overlapping, they will interfere with each other as is the case for individual having normal hearing. Thus, embodiments of the present invention so configured with multiple sound input elements and parallel and separate processing and stimulation using multiple stimulation modules as described above, individuals may be able to receive and distinguish between multiple conversations or other source sources.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A multi-mode hearing prosthesis for enhancing the hearing of a recipient, comprising:
a sound input element configured to receive a sound signal component;
a frequency spectral analysis module configured to analyze the sound signal component and to categorize the component into at least a high- or lower-frequency component;
a bone conduction processor configured to generate bone conduction stimulation signals from at least one of said high- and lower-frequency component for bone conduction stimulation of the recipient's skull; and
a second stimulation processor configured to generate auditory stimulation signals from at least one of said high- and lower-frequency components for stimulating the recipient.

2. The multi-mode hearing prosthesis of claim 1, wherein said second stimulation processor is an acoustic stimulation processor configured to generate amplified acoustic stimulation signals from at least one of said high- and lower-frequency component for amplified acoustic stimulation of the recipient.

3. The multi-mode hearing prosthesis of claim 1, further comprising a transducer communicably coupled to said bone conduction processor and configured to convert the at least one of said high- and lower-high-frequency component into mechanical force.

4. The multi-mode hearing prosthesis of claim 3, further comprising an implanted bone anchor mechanically coupled to said transducer and configured to transmit the bone conduction stimulation force from said transducer into the recipient's skull bone.

5. The multi-mode hearing prosthesis of claim 4, further comprising a coupler configured to mechanically couple said transducer to said implanted bone anchor.

6. The multi-mode hearing prosthesis of claim 3, wherein said transducer comprises one or more piezoelectric elements configured to generate said mechanical force.

7. The multi-mode hearing prosthesis of claim 3, wherein said mechanical force is generated parallel to the surface of the recipient's bone.

8. The multi-mode hearing prosthesis of claim 3, wherein said implanted bone anchor is configured to be positioned at least partially in the recipient's skull, and further configured to osseointegrate with the recipient's skull over a period of time.

9. The multi-mode hearing prosthesis of claim 2, further comprising an acoustic stimulation output module having an in-the-canal speaker positioned at least partially within the recipient's ear canal and configured to acoustically deliver said amplified acoustic stimulation to the recipient.

10. The multi-mode hearing prosthesis of claim 1, wherein said second stimulation processor is a direct acoustic stimulation processor configured to generate mechanical forces for directly manipulating the cochlear fluid in the cochlea of the recipient.

11. The multi-mode hearing prosthesis of claim 1, wherein said second stimulation processor is a mastoid stimulation processor configured to generate mechanical forces for directly stimulate the mastoid proximate the cochlea of the recipient.

12. A multi-mode hearing prosthesis for enhancing the hearing of a recipient, comprising:
a first sound input element configured to receive a high-frequency sound signal component;
a second sound input element configured to receive a lower-frequency sound signal component;
a bone conduction processor, configured to process said high-frequency sound signal component from said first input element and further configured to generate bone conduction stimulation to stimulate the recipient via bone conduction stimulation; and
a second stimulation processor configured to process said lower-frequency sound signal component from said second input element and further configured to generate stimulation signals to stimulate the recipient via a second stimulation mode,
wherein each of said first and second stimulation processors are configured to process said first and second signal components simultaneously.

13. A method for rehabilitating the hearing of a recipient with a multi-mode hearing prosthesis having two or more stimulation modules, comprising:
receiving an electrical signal representative of an acoustic sound signal;
analyzing said sound signal to generate at least a high-frequency component and a lower-frequency component from said acoustic sound signal;
delivering said high-frequency component via bone conduction to the recipient's skull bone; and
deliver said lower-frequency component via acoustic stimulation to the recipient's hearing organ.

14. The method of claim 13, wherein said delivering said high-frequency component comprises generating a mechanical force representative of said high-frequency component via a transducer and communicating said mechanical force to the recipient's skull bone.

15. The method of claim 14, wherein said communicating said mechanical force is via an implanted bone anchor communicably coupled to said transducer and configured to transmit the mechanical force from said transducer into the recipient's skull bone.

16. The method of claim 15, further comprising mechanically coupling via a coupler said transducer to said implanted bone anchor.

17. The method of claim 14, wherein said transducer comprises one or more piezoelectric elements configured to generate said mechanical force.

18. The method of claim 14, wherein said mechanical force is generated parallel to the surface of the recipient's bone.

19. The method of claim 15, further comprising positioning said implanted bone anchor at least partially into the recipient's skull, and allowing said anchor to osseointegrate with the recipient's skull over a period of time.

20. The method of claim 13, wherein said acoustic stimulation is delivered via an in-the-canal speaker positioned within the recipient's ear.

21. A multi-mode hearing prosthesis for enhancing the hearing of a recipient having two or more stimulation modules, comprising:
means for receiving an electrical signal representative of an acoustic sound signal;
means for analyzing said sound signal to generate at least a high-frequency component and a lower-frequency component from said acoustic sound signal;
means for delivering said high-frequency component via bone conduction to the recipient's skull bone; and
means for deliver said lower-frequency component via acoustic stimulation to the recipient's hearing organ.

22. The multi-mode hearing prosthesis of claim 21, wherein said means for delivering said high-frequency component comprises generating a mechanical force representative of said high-frequency component via a transducer and communicating said mechanical force to the recipient's skull bone.

23. The multi-mode hearing prosthesis of claim 21, wherein said means for deliver said lower-frequency component via acoustic stimulation to the recipient's hearing organ is an in-the-canal speaker positioned within the recipient's ear.

24. A method of stimulating a recipient with a multi-mode hearing prosthesis, comprising:
receiving a high-frequency sound signal component at a first sound input element;
receiving a lower-frequency sound signal component at a second sound input element;
processing said high-frequency sound signal component with a bone conduction processor configured to generate and deliver bone conduction stimulation; and
processing said lower-frequency sound signal component with a second stimulation processor configured to generate and deliver acoustic stimulation via a second stimulation mode,
wherein said bone conduction processor and second stimulation processor operate substantially concurrently.

25. The method of claim 24, wherein said second stimulation processor is an acoustic hearing aid stimulation processor configured to generate acoustic hearing aid stimulation signals.

26. The method of claim 24, wherein said second stimulation processor is a direct acoustic stimulation processor configured to generate direct acoustic stimulation signals.

27. The method of claim 24, wherein said second stimulation processor is a mastoid stimulation processor configured to generate mastoid stimulation signals.
